(19) United States
(12) Patent Application Publication
(10) Pub. No.: US 2013/0150902 A1
(43) Pub. Date: Jun. 13, 2013

(54) BLOCKED BONE PLATE AND SCREW PROVIDED WITH A THREADED CONIC OR PARABOLIC HEAD AND ARRANGEMENT COMPRISING AN IMPLANT AND RESPECTIVE INSERTION METHOD OF SAID IMPLANT TO A BONE TISSUE

(71) Applicant: Irineu Vitor LEITE, Curitiba (BR)

(72) Inventor: Irineu Vitor LEITE, Curitiba (BR)

(73) Assignee: NEOORTHO PRODUTOS ORTOPEDICOS, Curitiba (BR)

(21) Appl. No.: 13/348,976

(22) Filed: Jan. 12, 2012

(30) Foreign Application Priority Data
Dec. 13, 2011 (BR) 015110002713

(51) Int. Cl. A61B 17/80

(52) U.S. Cl. 606/291

(57) ABSTRACT

Blocked bone plate, provided with orthogonal bore holes, synergically associated to screws with a conic or parabolic head, threaded in said plate orthogonal bore holes, indicated for the treatment of fractures in part of the skeleton that, when affixed to a fractured bone, stabilizes the fracture. The nature of fixture of such a conic or parabolic head into an orthogonal bore hole is advantageous because it allows these fixture elements to perform micro movements that stimulate said bone consolidation.

### APPLICATION FIELD

The present invention relates to the field of medical apparatuses, more particularly the devices used in orthopedic treatment—implants, more specifically a blocked bone plate, aimed for the treatment of bone fractures, wherein its association with conic- or parabolic-headed screws permits screw micro movements, thus speeding up bone consolidation.

### BACKGROUND OF THE INVENTION

Implants are devices inserted into human body to perform the functions of some biological structure missing or with its functioning impaired. Such devices are well-known in art and since the use of conventional plates has been popularized by AO Group in the 1960s, they proved to be extremely useful for bone stabilization.

Ideally, the bone-plate-screw system should allow restoring member mechanical axle alignment, and restoring articular congruence to less than 2 mm deviation.

The first bone plates to be manufactured were the conventional fixture plates that might function as fixed systems only under optimal circumstances. These plates had simple bore holes housing a screw and compressing the plate towards the bone.

Then, a dynamic compression bone plate was introduced in 1969. The dynamic compression plate had a bore hole design allowing axial compression by screw eccentric insertion. The plate functions in different ways: compression, neutralization, tension band, or as a support.

Since during the use of dynamic compression plate and conventional plate there was the compression between plate and bone, periosteum capillary network was too harmed, leading to a relative worsening of cortical perfusion. So, dynamic compression bone plates were invented, with limited contact, which is an evolution from DCPs, where the area of contact plate-bone (the plate “gripping”) is gradually reduced.

In the early 90s, an experimentation of various types of inner fixtures for fractures was started. The initial focus was on developing stable fixtures that would not need large dissection of soft tissues.

So, there was a feeling of safety in using blocked plates. Blocked plates allow the insertion of screws or smooth pins with stable fixed angle. Screw bore holes had a threading allowing connection with the plate working as a fixed angle device. The relatively easy application and low incidence of complication reported in initial clinical trials contributed for spreading this technology for fracture fixtures.

The main biomechanical difference between blocked bone plates and conventional bone plates is that in the latter the stability occurs by friction between plate and bone, and system fixation occurs only by screw torque in the bone. With the persistence of physiological movements, the screws might loosen as the friction and looseness forces of the screw and, consequently, the plate diminish.

According to Jagodzinski and Krettek (2007), during the initial period, osteosynthesis should facilitate fragment micro movements i f fracture healing during second stage is desired. At the same time, articular congruence and axial and rotational positions should be maintained. During final healing stage, movement inside callus should be limited, which is naturally achieved by increased callus ossification rigidity.

Therefore, it is noticed in the technique a need for implants allowing micro movements of fixation elements (i.e., screws), thus taking less time for bone consolidation.

Based on that, and considering continuous product development, an invention is proposed claiming protection privileges for its novelty and inventive activity, as shown below.

A blocked bone plate is a plate which, when affixed to a fractured bone, provides support and/or stabilizes a fracture. In such a device, implant rigidity contributes to reduce fracture mobility. The absolute stability reduces tension in fracture so as to allow straight consolidation without visible calluses.

Also, the nature of fixture in such a conic or parabolic head screw into an orthogonal bore hole is advantageous because it allows these fixture elements to perform micro movements that stimulate said bone consolidation.

Prior Technique Analysis

US patent 20040006346 describes a screw implant for insertion into a bore hole arranged in bone tissue, said implant having a cancellous portion, and a cortical portion having an axial length such that, when installed in the bore hole, the engagement of said cortical portion with the bone tissue will generally be confined to the cortical bone tissue layer. In an alternative embodiment, it presents screws provided with a conic head, however threaded in an also conic bore hole, differently from the proposed invention.

US application 20100185246 reveals bone screws having heads which are provided with a hex socket as well as a threaded portion, allowing driving the screw through a plate system into a bone until desired compression is obtained. In an alternative embodiment, it protects blocking screws provided with a threaded conic head; however not associated to orthogonal bore holes, thus having none of the characteristics featured by the present invention.

U.S. Pat. No. 5,002,544 presents an osteosynthetic pressure plate having several holes for bone screws positioned along its longitudinal axis and having an underside with curved recesses, designed for positioning on the bone so that immediately upon implantation there are spaces between the bone and the pressure plate. It features bone implants provided with oblong holes for dynamic compression screws reliefs in its lower portion, however it does not mention conic or parabolic head screws threaded into orthogonal bore holes.

The invention EP1949866 describes an orthopedic bone implant and, more particularly, orthopedic bone plates having screw receiving apertures formed therein. In one exemplary embodiment, the implant includes an aperture defined by an interior threaded portion configured to engage the head of a bone screw. Such protection discloses bone plates provided with blocking screws with threaded conic head, but not threaded with orthogonal bore holes, thus not featuring the characteristics proposed by the present invention.

### BRIEF DESCRIPTION OF FIGURES

FIG. 1 shows side views of the screw provided with conic or parabolic head (1), wherein the distinction between conic or parabolic head threading (1) and fixation axle threading (2) is noticed.

FIG. 2 reveals a section view of the blocking screw (4) in bone plate (5), wherein the interaction (3) between screw head and bone plate and screw's long body (4) detaching from bone plate (5) is noticed.

FIG. 3 depicts a detail of screw head (1) threaded to the bone plate (5) and the interaction (3) between screw head and bone plate.

FIG. 4 depicts a detail of the interaction between screw threading (1) with a conic or parabolic head and the plate, when inserted thereto.

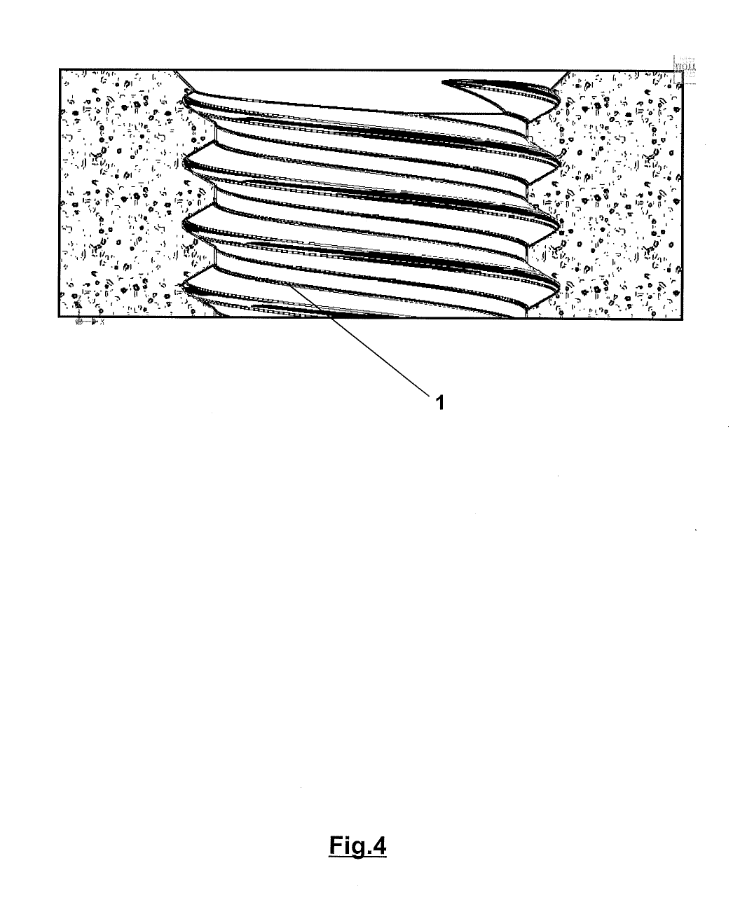

FIGS. 5A and 5B show a section view of the sequence of oscillation movements that the screw with a conic or parabolic head may perform when implanted.

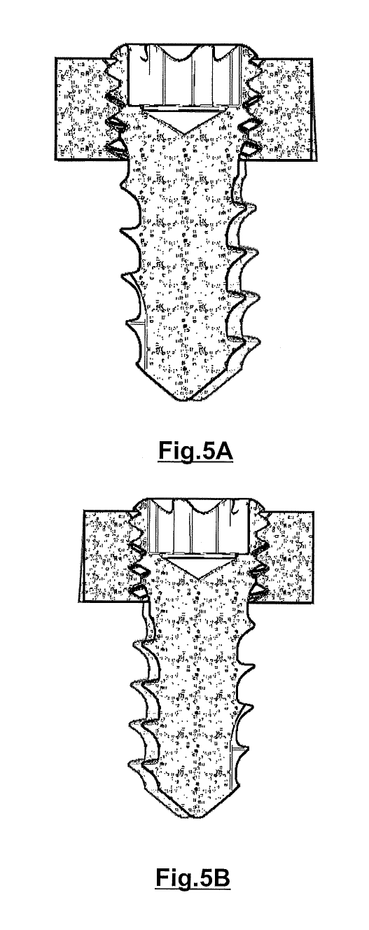

### DETAILED DESCRIPTION OF THE INVENTION

Facing the problems presented by prior art, the present invention aims at solving various existing problems in current technique by providing implant users with notable advantages to be detailed below, through an arrangement comprising a bone implant and method for inserting said implant into a bone tissue, thus allowing screw micro movements that speed up bone consolidation.

It is disclosed, therefore, a blocked bone plate provided with screws having conic or parabolic head to be threaded to an orthogonal bore hole (in a right angle) in the plate, aimed for treating fractures in any part of the skeleton.

A blocked bone plate allows micro movements resulting in screw (4) micro flexible fixation by interacting elements with different profiles, thus reducing tutor rigidity (i.e. the system that is directed to assist, restrict, align, or simulate the function of a body part, in this case, the plate-screw system), thus promoting a small relative stability in an inner fixation system.

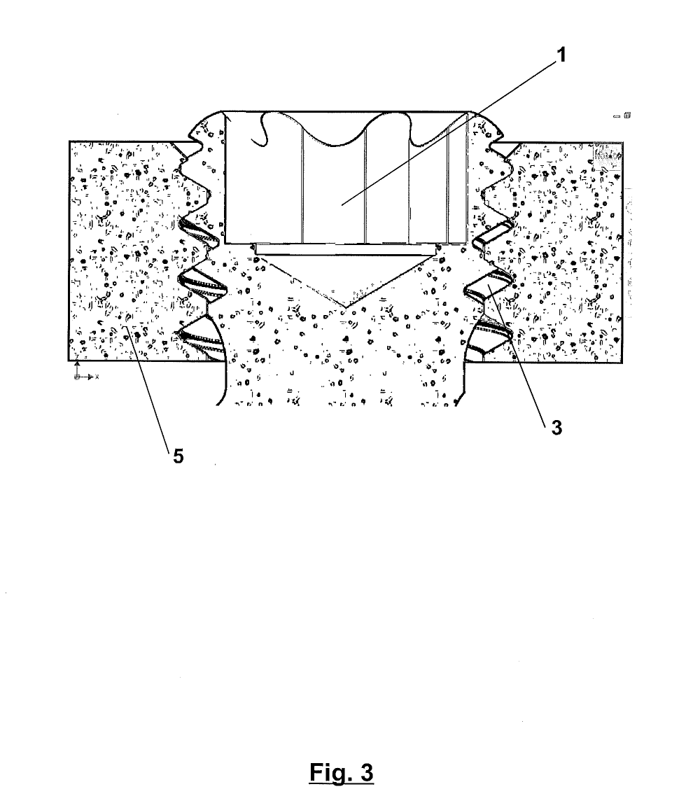

With different profiles, the screw (4) with conic or parabolic head (1) does not interact in the whole extension with plate orthogonal bore hole (5). The threading, although compatible, does not match the same way all over the longitudinal extension, since the different profiles are distant from each other, as seen in FIG. 3.

In traditional interactions, with orthogonal profiles, e.g., between a screw and a compatible nut, as threading advances the contact area between elements increase, since identical profiles cause equidistance between screw walls and threads as threading advances. In the present invention, the parabolic profile (1) of the screw head (4) provides an interaction zone for fixation only in the upper part, as depicted in FIG. 3. The remainder of screw body is “loose” inside plate bore hole cavity (5) due to the walls growing distant as the conic or parabolic head (1) diameter diminishes.

Additionally, the recesses located in plate inner surface (5), that are in contact with the bone, surrounding the bore hole, allow more space for screw movements (4), thus allowing more micro movements in screw body (4).

The plate, besides having blocked bore holes, is also provided with dynamic compression bore holes of limited contact, allowing both the use of a conventional screw and a blocked screw. This combination reflects the medical need of more flexibility in plate osteosynthesis, i.e., depending on fracture condition the conventional technique can be applied; compression technique, bridge compression method, inner fixture principle, or a combination of both principles.

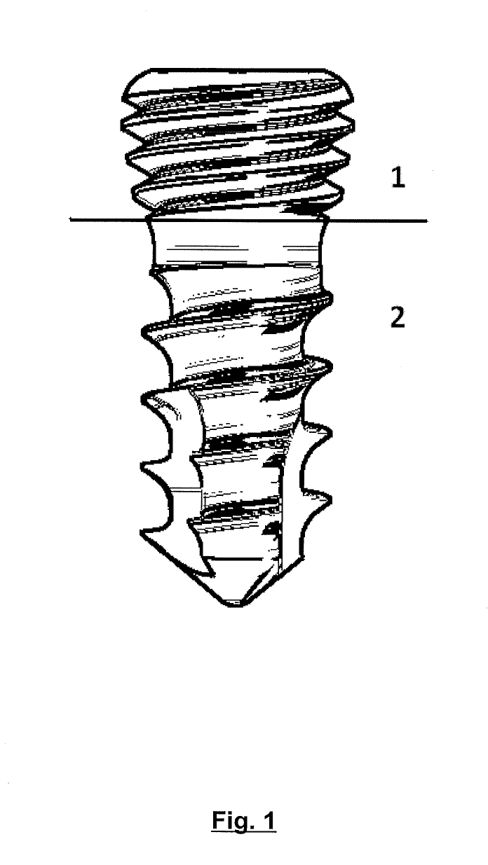

The plate is characterized by the fact that inner jacket surface is orthogonal, i.e., a threaded bore hole with a right angle, with parallel walls. The screw (4) is characterized by 2 regions: region 1 having a head a threaded with parabolic profile, said head having its diameter diminished as the threading advances screw body; such region 1 is blocked in plate orthogonal bore hole. In region 2, the screw threading is affixed to the bone, which is best understood by seeing FIG. 1.

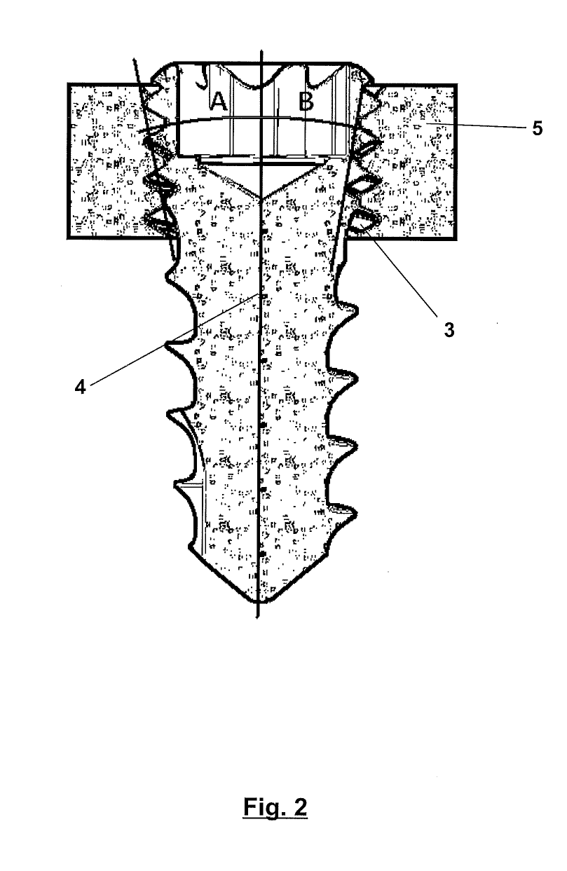

The threading in region 1 has a conic or parabolic outer surface in an angle “A” ranging from 2° to 25° and a conic or parabolic inner surface in an angle “B” ranging from 2° to 25°, as shown in detail in FIG. 2.

The screw is blocked to the plate by using a controlled torque varying according to threading measures. During this assembly, the screw is locked in one of the bore hole upper threads, thus keeping bore hole lower region from contacting screw threading, and allowing the space necessary for micro movements, as seen in FIG. 3.

The lower region of the plate and screw locking system, shown in FIG. 2, which is not in contact during application due the distance between the walls of the screw with conic or parabolic head (1) and plate bore hole (5), allows micro movements due to screw (4) bending, once this is also affixed to the bone.

This invention is not limited to the representations commented or illustrated herein, and it should be understood in its wide scope. Many modifications and other representations of the invention shall be provided by those skilled in the technique, with the benefit of the teachings presented in previous descriptions and annexed drawings. Besides, it should be understood that the invention is not limited to the embodiments disclosed herein, and that modifications and other embodiments are construed as within the scope of the annexed claims. Even though specific terms are applied herein, they should be construed only for generic and descriptive purposes and not for limiting purposes.

What is claimed is:

1. Arrangement with blocked bone plate and screw provided with a threaded conic or parabolic head, characterized by being applied in association, interacting so as to allow micro movements of the screw (4) by flexing its axle, improved by recesses of the contact surface with the bored plate with the bone, in the region surrounding the orthogonal threaded bore holes.
